(19) United States
(12) Patent Application Publication
(10) Pub. No.: US 2015/0157253 A1
(43) Pub. Date: Jun. 11, 2015

(54) SYSTEM AND METHOD FOR HUMAN AGE ESTIMATION BASED ON IN VIVO SKIN IMAGING

(71) Applicant: NATIONAL TAIWAN UNIVERSITY, Taipei (TW)

(72) Inventors: Chi-Kuang SUN, Taipei (TW); Yi-Hua LIAO, Taipei (TW)

(73) Assignee: NATIONAL TAIWAN UNIVERSITY, Taipei (TW)

(21) Appl. No.: 14/098,550

(22) Filed: Dec. 6, 2013

(51) Int. Cl. A61B 5/00

(52) U.S. Cl. CPC A61B 5/441; A61B 5/0068

(57) ABSTRACT

A system for human age estimation based upon in vivo skin imaging includes a harmonic generation microscopy (HGM) device and an analysis device. The HGM device is configured for observing human skin and forming sectioned images of the human skin of second harmonic generation (SHG), third harmonic generation (THG), and combination of the SHG and the THG of an excitation light scanning the human skin. The analysis device is in communication with the HGM device and configured for processing the sectioned images to reveal relationships between the morphological features of the human skin and chronological age of a human being having the human skin.

### BACKGROUND

1. Technical Field

The present disclosure relates to human age estimation and identification technologies and particularly to a system and method for estimating human chronological age based upon in vivo skin imaging thereof.

2. Description of Related Art

At present, to estimate human chronological age, bone or dental X-ray imaging methods are employed to obtain human bone or dental age which in turn is used to indicate the human chronological age. However, the X-ray imaging methods are harmful due to the exposure to the radioactive X-ray.

U.S. Pat. No. 6,922,279 which has a same co-inventors with the present disclosure and assigned to the same assignee as the present disclosure discloses a harmonic generation microscopy for providing an image of a biological sample in vivo. No further disclosure in the '279 patent discusses the use of the microscopy to determine human age based on in vivo skin imaging. US Patent Application Publication No. 2013/0001408 A1 which also has a same co-inventors with the present disclosure and assigned to the same assignee as the present disclosure discloses a further improvement over the '279 patent. However, the '408 patent publication still does not disclose the use of the microscopic system as a device for determining human age based on in vivo skin imaging.

Therefore, it is desirable to provide a system and method to estimate human chronological age that can overcome the above-mentioned problems.

### BRIEF DESCRIPTION OF THE DRAWINGS

Many aspects of the present disclosure can be better understood with reference to the following drawings. The components in the drawings are not necessarily drawn to scale, the emphasis instead being placed upon clearly illustrating the principles of the present disclosure.

FIG. 1 is a schematic view of a system for estimating human chronological age based on in vivo skin imaging, according to an embodiment.

FIG. 2 is a schematic view showing how the system of FIG. 1 is used to perform a scanning operation on human skin.

FIG. 3 schematically shows exemplary sectioned images of the human skin.

FIG. 4 schematically shows layers of the human skin.

FIG. 5 schematically shows layers of the epidermis of the human skin.

FIG. 6 schematically shows an isolated papillae volume obtained by an analysis device of the system of FIG. 1 for an analysis of the skin in vivo.

FIG. 7 shows graphs of statistical results indicating a relationship between a height of the papilla and the human chronological age.

FIG. 8 shows graphs of statistical results indicating a relationship between a volume of the papilla and the human chronological age.

FIG. 9 shows graphs of statistical results indicating a relationship between a thickness of the epidermis including the papillae and the human chronological age.

FIG. 10 schematically shows a diagram of a junction between the epidermis and the denims obtained by the analysis device of FIG. 1 for another analysis of the skin in vivo.

FIG. 11 shows graphs of statistical results indicating a relationship between a 3D interdigitation index and the human chronological age.

FIG. 12 shows schematic sectioned images of the stratum basale of different subjects.

FIG. 13 shows graphs of statistical results indicating a relationship between a cellular area of the stratum basale and the human chronological age.

FIG. 14 shows graphs of statistical results indicating a relationship between a nucleus area of the stratum basale and the human chronological age.

### DETAILED DESCRIPTION

Embodiments of the present disclosure will be described with reference to the drawings.

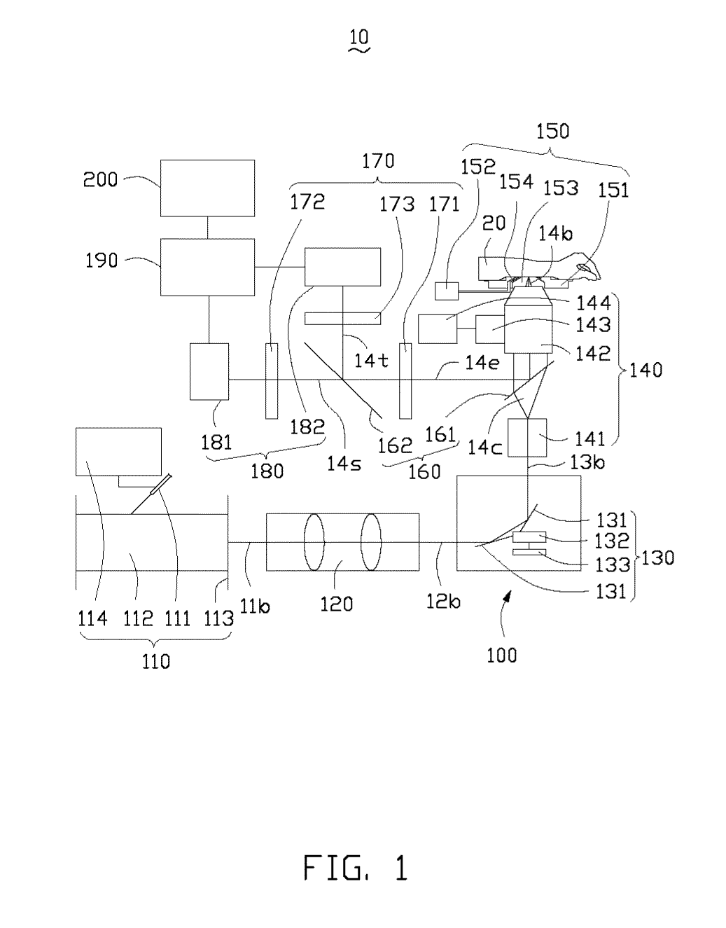

Referring to FIG. 1, a system 10 for human age estimation based on in vivo skin imaging, according to an embodiment, is shown.

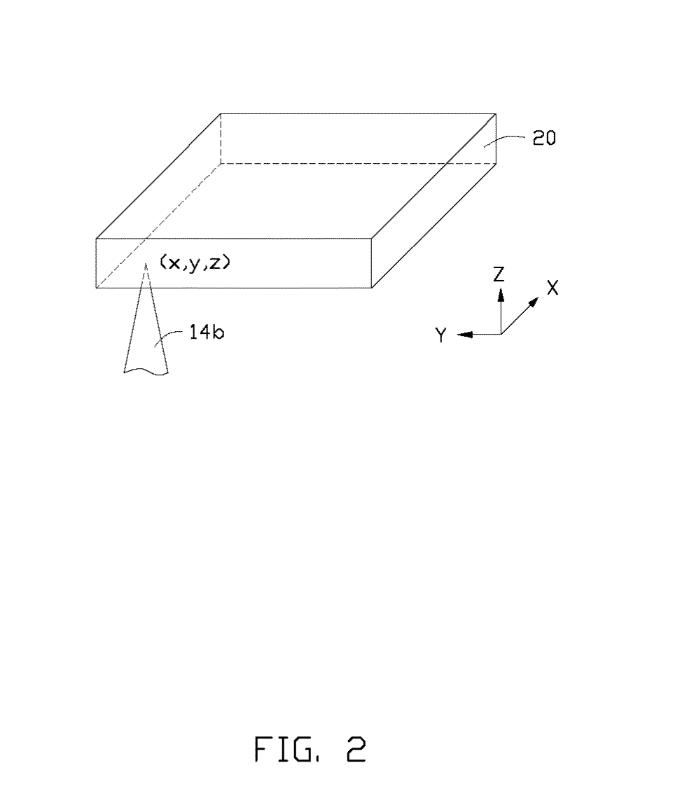

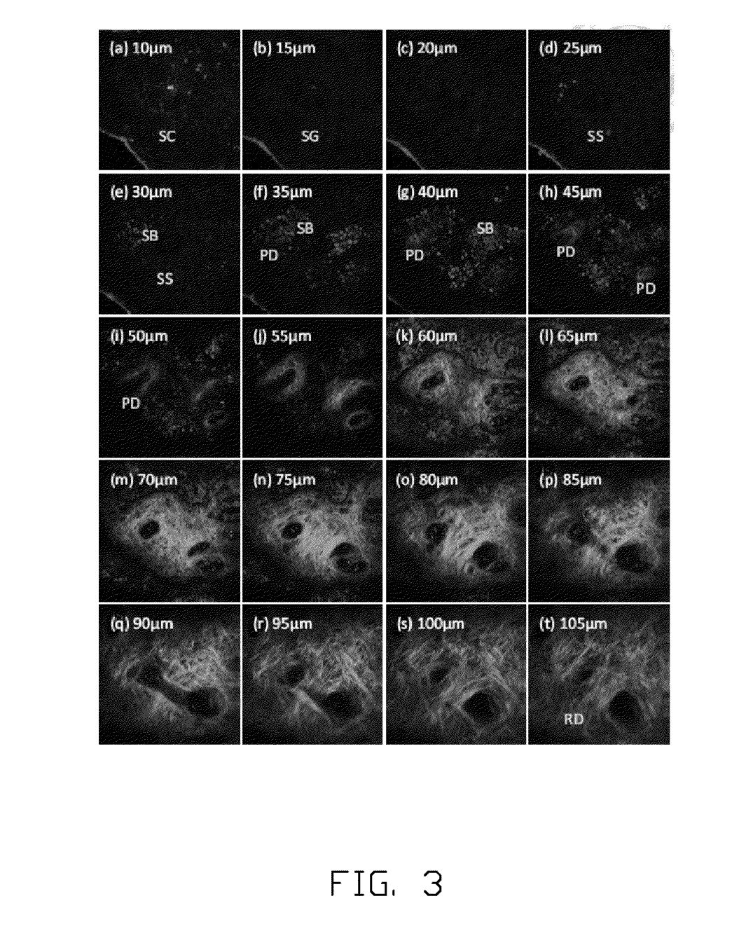

The system 10 includes a harmonic generation microscopy (HGM) device 100 configured for observing human tissues such as skin of leg, forearm, abdomen, and any other suitable portion, whose aging is caused by intrinsic factor, i.e., chronological age. The HGM device 100 is also configured for forming sectioned images of the human skin (see FIG. 3) of harmonic generations, such as second harmonic generation (SHG) and third harmonic generation (THG), of an excitation light that scans the human skin (see FIG. 2). In this embodiment, the SHG and THG are used. However, in other embodiment, a fourth harmonic generation can be used.

The system 10 also includes an analysis device 200. The analysis device 200 is in communication with the HGM device 100 and configured for processing the sectioned images and thus revealing relationships between morphological features of the human skin and human chronological ages.

The relationships can be stored as a database. The analysis device 200 can also be configured for processing the sectioned images of a sample 20 of a person (e.g., the human skin of the ventral forearm about 15 centimeters (cm) of the wrist of the person) to obtain the morphological features of the sample 20 and thus to assist to estimate the chronological age of the person using the database, i.e., the revealed relationships.

In principle, skin aging is a process of structural integrity loss and physiological changes caused by both intrinsic and extrinsic factors. Intrinsic aging of skin is a natural process genetically determined and inalterable. Morphologic changes related to intrinsic aging in older skin are relatively subtle and consist of primary laxity, fine wrinkling, and a variety of benign neoplasms. The changes can be observed and investigated in cellular level and used to estimate the human chronological age.

It is noteworthy that results of the present in vivo skin imaging are also useful for evaluations of skin health and/or anti-aging effects of cosmetic products and/or therapeutic methods considering that many cosmetic products and therapeutic methods are proposed to restore the human skin toward a younger age. The results can also be used for dermatological diagnoses.

As a non-invasive tool, only virtual-level transition is involved in the in vivo skin imaging and thus no energy is left in the human skin. Accordingly, no photodamage is caused to the human skin.

In practice, the observation of the human skin is limited to a certain time, such as up to about 30 minutes, and average power applied to the human skin is controlled within a safety range, such as under about 100 milliwatts (mW). It is to be noted that the time and the power are subject to change according to the tested patients' tolerance and patience. Experimental results show that the in vivo skin imaging is comfortable and does not cause any itch or pain.

In addition, no dye is needed because that the human skin is observed by the HGM device 100, whereby damage to the human skin occurring in conventional skin biopsy methods is also eliminated.

The HGM device 100 includes a laser device 110, a beam shaping device 120, a scanning device 130, an objective lens device 140, a holding device 150, a beam splitting device 160, a filtering device 170, a detecting device 180, and an image processing device 190.

The laser device 110 is configured to emit a laser beam 11b with a predetermined wavelength, pulse width, repetition rate, and output power. The predetermined wavelength falls into a range from about 1200 nanometers (nm) to 1330 nm, such as about 1230 nm, 1250 nm, and 1260 nm. The predetermined pulse width falls into a range from about 15 femtoseconds (fs) to about 500 fs, such as about 51 fs, 100 fs, and 200 fs. The predetermined repetition rate falls into a range from about 10 million hertz (MHz) to about 150 MHz, such as about 50 MHz, 80 MHz, 95 MHz, and 110 MHz. The predetermined output power falls into a range from about 10 milliwatts (mW) to about 1500 mW, such as about 320 mW, 500 mW, 580 mW, and 800 mW.

The laser beam 11b is processed into the excitation light by the beam shaping device 120, the scanning device 130, and the objective lens device 140.

By employing the predetermined wavelength, the pulse width, the repetition rate, and the output power, the section images of the SHG, THG or combination of SHG and THG of the excitation light are clear (i.e., of relatively high resolution and contrast) and distinct, the output power applied to the human skin is controlled to the safe range, and a penetration depth of the excitation light can be greater than about 300 microns (um) or even approach about 1 millimeter (mm).

In this embodiment, the laser device 110 includes a pump source 111, a gain medium 112, an optical cavity 113, and a laser controller 114.

The pump source 111 is configured for supplying energy to the gain medium 112 in a form of electrical current or pumping light having a wavelength different from the predetermined wavelength. The gain medium 112 is configured for amplifying light of the predetermined wavelength. The optical cavity 113 is configured for bouncing the amplified light back and forth, passing through the gain medium 112, such that the amplified light is amplified each time passing the gain medium 112 and comes out as the laser beam 11b. The laser controller 114 is in communication with the pump source 111, the gain medium 112, and the optical cavity 113 and is configured to control various parameters of the laser beam 11b, such as the wavelength, the pulse width, the repetition rate, and the output power.

The pump source 111 can be but is not limited to a semiconductor laser, an ytterbium fiber laser with a wavelength at about 1060 nm, a neodymium doped yttrium aluminum garnet laser, a titanium doped sapphire laser with a wavelength at about 800 nm, or a large-mode-area ytterbium doped photonic crystal fiber (PCF) laser with a wavelength of about 1040 nm, a repetition rate of about 50 MHz, and a pulse width of about 100 fs.

The gain medium 112 can be but is not limited to chromium doped forsterite.

The optical cavity 113 can be but is not limited to an optical parametric oscillator (OPO) or a PCF oscillator. The optical cavity 112 can include optical element of special designs such as curvatures, coatings for obtaining the predetermined parameters of the laser beam 11b such as the predetermined wavelength and pulse width.

In this embodiment, the laser source 11 can be a femtosecond chromium doped forsterite laser with the predetermined wavelength of about 1230 nm, the predetermined pulse width of about 100 fs, the predetermined repetition rate of about 110 MHz, and the predetermined output power of about 500 mW pumped by the about 1060 nm ytterbium fiber laser.

Alternatively, the laser source 11 can be a femtosecond chromium doped forsterite laser with the predetermined wavelength of about 1260 nm, the predetermined pulse width of about 51 fs, the predetermined repetition rate of about 95 MHz, and the predetermined output power of about 800 mW pumped by the about 1060 nm ytterbium fiber laser.

In another embodiment, the laser source 11 can be a femtosecond OPO laser with the predetermined wavelength of about 1250 nm, the predetermined pulse width of 200 fs, the predetermined repetition rate of about 80 MHz, and the predetermined output power of about 580 mW, pumped by the about 800 nm titanium doped sapphire laser.

In further another embodiment, the laser source 11 can be a femtosecond laser with the predetermined wavelength of about 1260 nm, the predetermined repetition rate of about 50 MHz, the predetermined pulse width of about 100 fs, and the predetermined output power of 320 mW based on a 1040 nm, 50 MHz, 100 fs amplified output of the large-mode-area ytterbium-doped PCF oscillator-amplifier laser.

The beam shaping device 120 is positioned in a path of the laser beam 11b and configured for shaping the laser beam 11b into a shaped beam 12b having a shape, size, and collimation degree complying with requirements of the scanning. In this embodiment, the beam shaping device 120 can include collimator lenses for collimating the laser beam 11b.

In other embodiments, if the laser beam 11b already satisfies the requirement of scanning, the beam shaping device 120 can be omitted.

Also referring to FIG. 2, the scanning device 130 and the objective lens device 140 are positioned along a path of the shaped beam 12b and are configured for directing the shaped beam 12b as a scanning beam 14b which is focused into the human skin, e.g., the sample 20, by the objective lens device 140 to form a scanning point (x, y, z). The scanning device 130 and the objective lens 140 are configured for moving the scanning point (x, y, z) to perform a three-dimensional scanning operation on the human skin, e.g., the sample 20. The scanning beam 14b functions as the excitation light.

The scanning device 130 can include a pair of mirrors 131, a scanning motor 132, and a scanning controller 133.

The mirrors 131 are positioned along the path of the shaped beam 12b. The scanning motor 132 is coupled to the mirrors 131. The scanning controller 133 is in communication with the scanning motor 132 and configured for controlling the scanning motors 132 to realize fast and precise positioning of the mirrors 131 for deflection of the shaped beam 12b.

For example, one of the mirrors 131 can move the scanning point (x, y, z) along the X-direction of an XYZ coordinate system, and the other mirror 131 can move the scanning point (x, y, z) along a Y-direction of the XYZ coordinate system, thus realizing an XY-plane scanning operation within one of the stacks of the human skin, e.g., the sample 20. The XYZ coordinate system is established in such a manner that a Z-direction of the XYZ coordinate system is substantially perpendicular to a depth of the human skin. The Z direction movement is accomplished either by the displacement of the objective lens device 140 or by the sample 20 or by both of the mentioned. As such, by scanning each XY-plane, a sectioned image of each stack of the human skin can be formed.

The scanning motor 132 is typically based on moving magnet technology.

The scanning controller 133 is in communication with the scanning motor 132 and is configured for control various parameters of the scanning, such as a scanning range of the XY-plane, step sizes in the X-direction and the Y-direction, and a scanning speed. The scanning range of the XY-plane can be but not limited to about 120 um times about 120 um. That is, each sectioned image has a 120 um*120 um size. The step size in the X-direction and the Y-direction can both be but is not limited to about 120/512 um. That is, each sectioned image has 512*512 pixels. The scanning speed can be but is not limited to about 2.7/262144 second for each step. That is, the sectioned images have a frame speed of bout 2.7 seconds.

In this embodiment, the scanning device 130 can have a galvo pair scanning mirrors or galvo-resonance scanning mirrors schema and outputs a rotating beam 13b.

The objective lens device 140 includes a tube lens 141, an objective lens 142, an objective motor 143, and an objective controller 144.

The tube lens 141 and the objective lens 142 are positioned along the path of the rotating beam 13b. The tube lens 141 is configured for diverging the rotating beam 13b to be a light cone 14c, which is focused by the objective lens 142 into the human skin, e.g., the sample 20. That is, the objective lens 142 is configured for focusing the light cone 14c into the human skin, e.g., the sample 20. The objective motor 143 is coupled to the objective lens 142. The objective controller 144 is in communication with the objective motor 143 and configured for controlling the objective motor 143 to move the objective lens 142 along a direction that is substantially parallel with the Z-direction, thus moving the scanning point (x, y, z) along the Z-direction. As such, the sectioned images of different stacks of the human skin at different depths thereof can be obtained by moving the objective lens 142 after each XY-plane scanning operation.

In this embodiment, the objective lens 142 can be but is not limited to an infrared water immersion objective lens.

The objective controller 144 is configured for controlling various parameters of the scanning, such as a step size in the Z-direction and a scanning depth. The step size in the Z-direction can be about 5 um. The scanning depth can be about 300 um.

The holding device 150 is positioned adjacent to the objective lens 142 and configured for holding and stabilizing the human skin, e.g., the sample 20. During the present in vivo skin imaging, vibration of the human skin, e.g., the sample 20, due to, e.g., breathing, can lead to image blurring. As such, the holding device 150 is employed for stabilization of the human skin, e.g., the sample 20

In this embodiment, the holding device 150 includes a substantially disk-like main body 151 and a vacuum source 152. The main body 151 defines a substantially circular observation window 153 through a substantially central portion thereof. The main body 151 also defines a number of vents 154 surrounding the window 153. The objective lens 142 is inserted into the window 153. The vents 154 are communicated with the vacuum source 152.

As such, the human skin, e.g., the sample 20, can be vacuum-held by the vacuum source 152 through the vents 154 and is exposed to the scanning beam 14b from the objective lens 142 via the window 153 after being put on the main body 151.

It could be understood that the holding device 150 is not limited to this embodiment but can take other forms depending on needs. For example, in other embodiments, the human skin, e.g., the sample 20, can be stabilized by magnetic mechanisms or adhesive tapes.

For each scanning point (x, y, z), an observation beam 14e containing SHG 14s and THG 14t of the excitation light is induced and reflected off due to SHG and THG effects.

SHG and THG effects are nonlinear optical processes, in which photons interacting with a nonlinear material are effectively combined to form new photons with twice and triple the energy, and therefore twice and triple a frequency of photons of the excitation light.

SHG often occurs in a noncentrosymmetric and continuous structured media, especially nano-structures, such as stacked membranes, aligned protein structures, and microtubule arrays. However, SHG is not suitable for an observation of interfaces in biological samples.

Since all materials have non-varnishing third order coefficient and since the coefficient is different at different portions of an observed sample, which induces variation of THG intensity, THG is commonly used in non-linear scanning microscopic imaging process. Since THG often occurs in the interface, THG is not suitable for observation of bulk noncentrosymmetric media, which, however, can be clearly inspected by means of SHG.

As such, the SHG and the THG can be combined to observe entire structures of the biological samples.

The beam splitting device 160 is positioned in a path of the observation beam 14e and is configured for separating and directing the SHG 14s and the THG 14t to transmit separately to facilitate independent processing of the SHG 14s and the THG 14t. The beam splitting device 160 includes a first dichromatic beam splitter (DBS) 161 and a second DBS 162.

The first DBS 161 is positioned between the tube lens 141 and the objective lens 142 and configured for passing the light cone 14c directly but deflecting the observation beam 14e. As such, the observation beam 14e is separated from the light cone 14c for further processing.

The second DBS 162 is positioned in the path of the observation beam 14e reflected off from the first DBS 161 and is configured for passing the SHG 14s directly but deflecting the THG 14t. As such, the SHG 14s and the THG 14t are separated and directed to different directions for respective and independent processing.

In this embodiment, the first DBS 161 works at about 850 nm and the second DBS 162 works at about 490 nm and both are but is not limited to about 45-degree tilted in relative with a transmitting direction of the observation beam 14e.

However, configurations of the first DBS 161 and the second DBS 162 are not limited to this embodiment but can be changed depending on needs. For example, the first DBS 161 and the second DBS 162 can work at other wavelengths depending on needs. Further, the second DBS 162 can alternatively pass the THG 14t while reflect the SHG 14s.

The filtering device 170 includes a first filter 171 located in a path of the observation beam 14e and second and third filters 172, 173 positioned in paths of the SHG 14s and the THG 14t, respectively. The second and third filters 172, 173 are configured for filtering noises out of the SHG 14s and the THG 14t to facilitate further processing of the SHG 14s and the THG 14t, e.g., increasing signal-to-noise ratio of the sectioned images.

The first filter 171 is positioned between the first DBS 161 and the second DBS 162 and is configured for filtering noises out of the observation beam 14e consisting of the SHG 14s and THG 14t. The second filter 172 is positioned in the path of the SHG 14s from the second DBS 162 and configured for filtering noises out of the SHG 14s. The third filter 173 is positioned in the path of the THG 14t from the second DBS 162 and is configured for filtering noises out of the THG 14t.

In this embodiment, the first filter 171 works at wavelengths from about 400 nm to about 700 nm (e.g., a color filter), the second filter 172 works at wavelengths from about 600 nm to about 650 nm, and the third filter 173 works at wavelength from about 400 nm to about 430 nm (i.e., both are band-pass filters).

The specific wavelengths given above for the first filters 171, the second filter 172, and the third filter 173 are just for exemplarily illustrate the present disclosure and should not limited the scope of this invention.

Configuration of the filtering device 170 is not limited to this embodiment but can take other forms in other embodiments depending on needs.

The detecting device 180 is positioned in paths of the SHG 14s and the THG 14t and is configured for detecting and converting the filtered SHG 14s and the THG 14t of each scanning point (x, y, z) into electrical signals, such as electric charges, currents, and voltages, of values that are proportional to intensities of the SHG 14s and the THG 14t. The first detector 181 and the second detector 182 also can convert the electrical signals into digital signals by employing analog-to-digital (AD) technologies to facilitate further processing.

The detecting device 180 includes a first detector 181 and a second detector 182. The first detector 181 is positioned in the path of the SHG 14s from the second filter 172 and is configured for detecting the SHG 14s of each scanning point (x, y, z). The second detector 181 is positioned in the path of the THG 14t from the third filter 173 and is configured for detecting the THG 14t of each scanning point (x, y, z).

In this embodiment, the first detector 181 and the second detector 182 are photomultiplier tubes. However, configuration of the detecting device 180 is not limited to this embodiment but can take other forms in other embodiments depending on needs.

Referring to FIG. 1, the image processing device 190 is in communication with the scanning controller 133, the objective controller 144, and the detecting device 180 and configured for synchronizing the detecting device 180 with the scanning controller 133 and the objective controller 144. The image processing device 190 thus is capable of reading the electric signals or digital signals corresponding to the SHG 14s and the THG 14t of each scanning point (x, y, z) and plotting the sectioned images of the SHG 14s, the THG 14t, or combination of the SHG 14s and the THG 14t of each scanning point (x, y, z) with assistance of properties of the SHG 14s and the THG 14t (i.e., the SHG 14s is suitable for observation of bulk noncentrosymmetric media while the THG 14t is suitable for observation of interfaces).

In this embodiment, the sample 20 is the human skin of the forearm of a 24-year female. As shown in FIG. 3, the sectioned images of different stacks including the stratum corneum (SC), the stratum granulosum (SG), the stratum spinosum (SS), the stratum basale (SB), the papillary dermis (PD), and the upper reticular dermis (RD) are illustrated. Depths of the sectioned images are labeled.

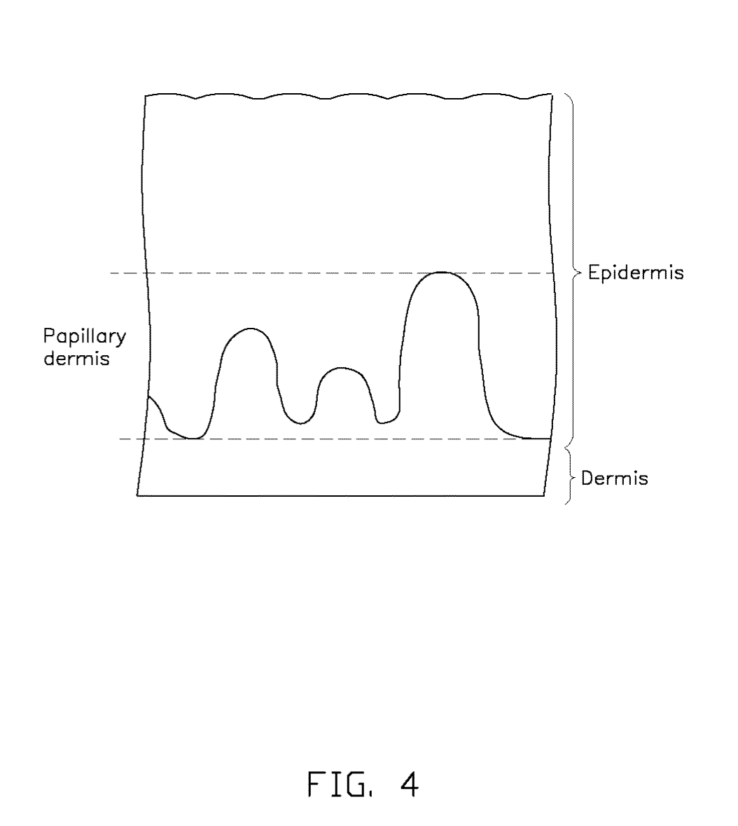

Referring to FIG. 4, briefly, the human skin includes, in this order from a superficial layer to a deep layer of human skin, the epidermis and the dermis. The dermis includes, in this order from the shallow to the deep layers, the papillary dermis and the upper reticular dermis. The papillary dermis includes a number of dermal papillae.

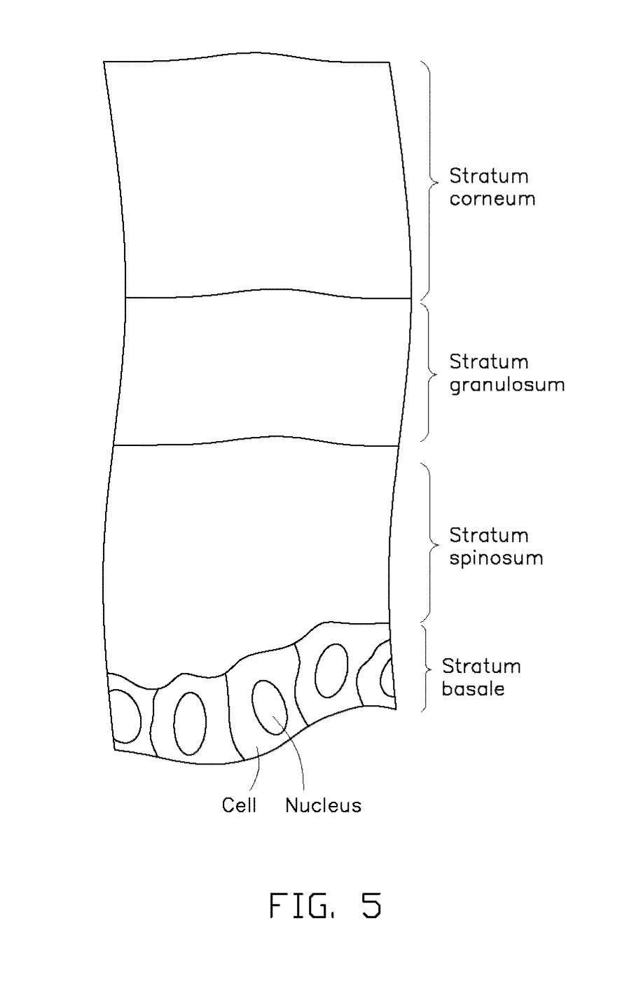

As referring to FIG. 5, in brief, the epidermis includes, form the superficial to the deep layers, the stratum corneum, the stratum granulosum, the stratum spinosum, and the stratum basale. The stratum basale includes a number of substantially honeycomb-like basal cells, each of which includes a nucleus.

To reveal the relationships of the morphological features of the human skins and human chronological ages, a population study is conducted by the system 10 on 48 Asian subjects with 7 females and 8 males aged 19-29 years, 13 females and 6 males aged 30-59 years, and 8 females and 6 males aged 60-79 years, of Fitzpatrick skin phototype III or IV. Each subject is observed by the HGM device 100 to obtain a corresponding group of sectioned images. The study is conducted according to the Declaration of Helsinki Principles, and the protocol is approved by the Institutional Review Board of National Taiwan University Hospital. Informal consent is obtained from each subject prior to the study.

Analyses on various morphological features including the following ones that indicate relationships between the morphological features and the human chronological ages are performed by the analysis device 200 by processing each group of sectioned images.

1. Height and Volume of Papilla

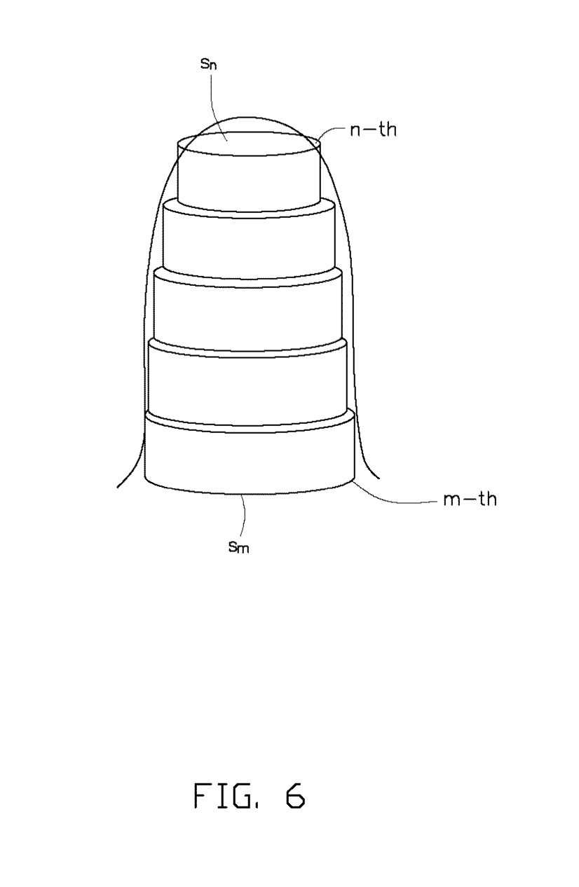

Referring to FIG. 6, for each group of sectioned images of the SHG 14s or combination of the SHG 14s and THG 14t, each papilla is recognized by processing the sectioned images, in this order from the superficial to the deep layers, and the n-th sectioned image in which the papilla is first present and the m-th sectioned image in which the papilla is first vanished are found by the analysis device 200, wherein n, m are positive integers and m>n. Then, a height of the papilla (i.e., isolated dermal papilla height) can be calculated by the formula: H=z*|(M−N)| using the analysis device 200, wherein H is the papilla height and z is the step size of the scanning in the Z-direction.

In addition, an area of the papilla in each sectioned image in which the papilla is present is measured by the analysis device 200. Then, a volume of the papilla (i.e., isolated dermal papilla volume) can be calculated by the formula:

$$V = {\sum }_{i = m}^{i = n} z * {s}_{i}$$

using the analysis device 200, wherein V is the volume and sᵢ is the area of the papilla in the i-th sectioned image, and N≦i≦m.

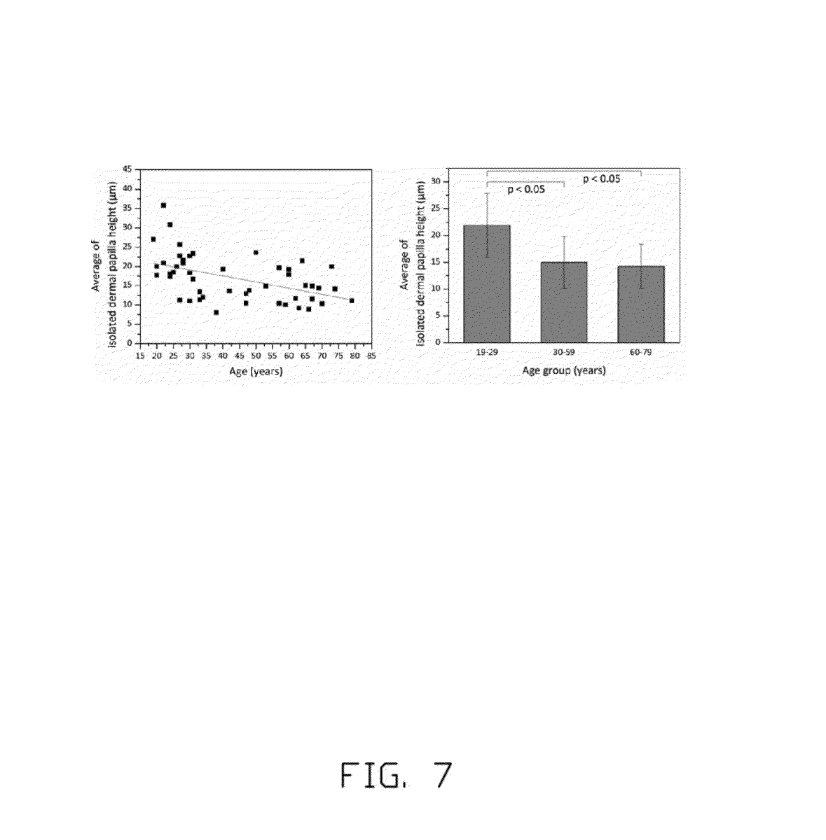

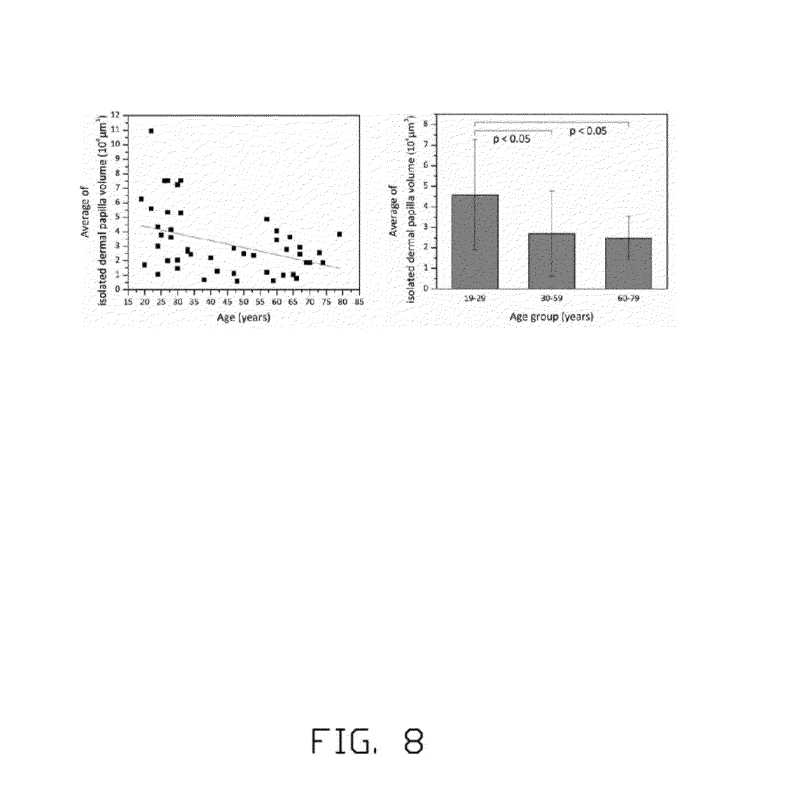

Referring to FIGS. 7-8, statistical results show that the average height and average volume of the papillae of the skin decrease with aging.

2. Thickness of Epidermis Including Papillae

Referring back to FIG. 5, for each group of sectioned images, the M-th sectioned image in which all the papillae are first vanished is found by the analysis device 200. A thickness of the epidermis including the papillae, i.e., viable epidermis (including the rete ridge), is calculated by the formula: T=M*z using the analysis device 200, wherein T is the thickness of the epidermis including the papillae and M is a positive integer.

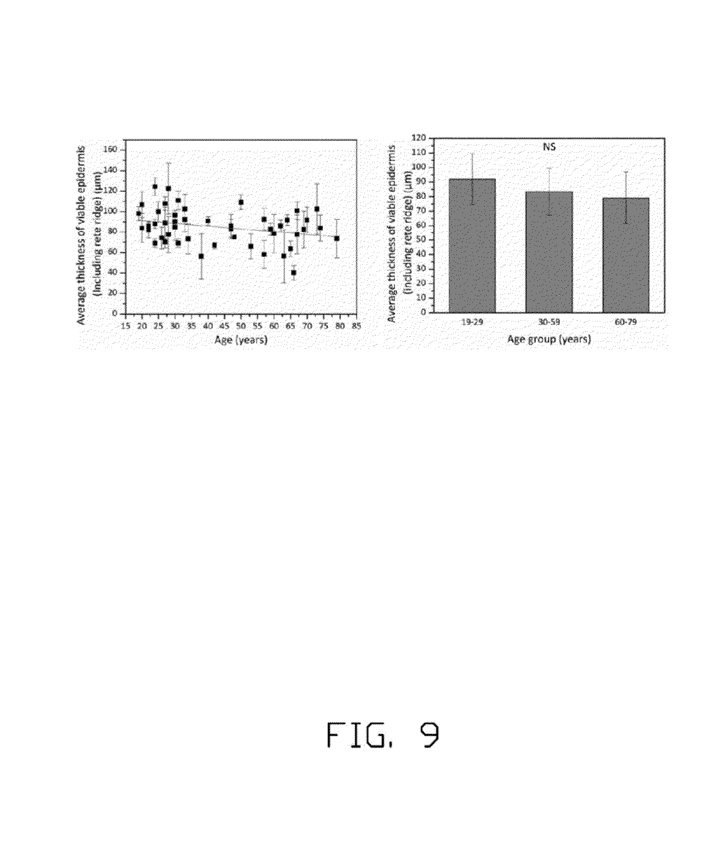

Referring to FIG. 9, statistical results show that the average thickness of the viable epidermis including the papillae of the skin decreases with aging.

3. Flatten of Epidermis-Dermis Junction

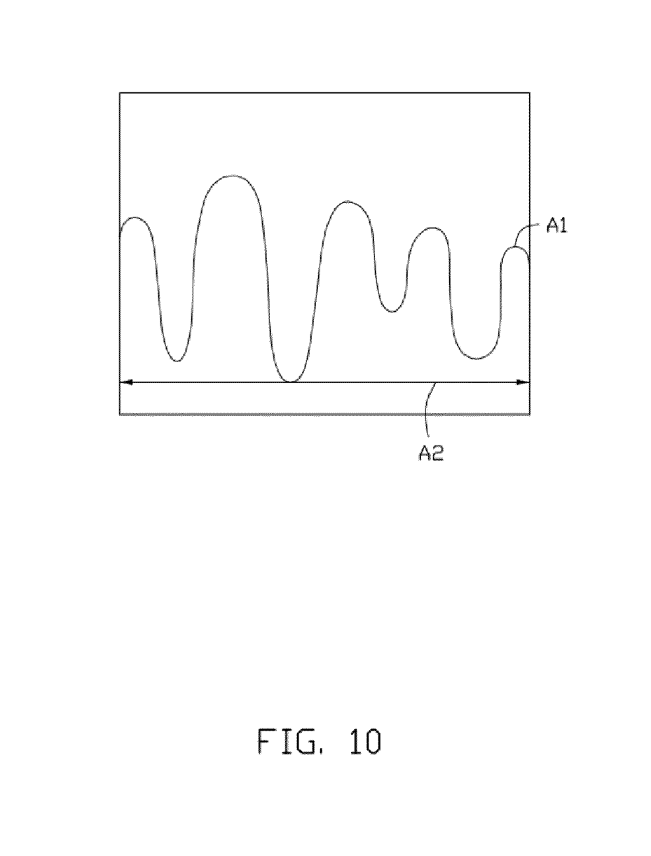

Referring to FIG. 10, for each group of sectioned images of the SHG 14s or combination of the SHG 14s and the THG 14t, the N-th sectioned image in which any papilla is first present is found by the analysis device 200. Then, an interface area is calculated by the formula:

$$A 1 = {\sum }_{i = M}^{i = N} z * {L}_{i}$$

using the analysis device 200, wherein Lᵢ is a circumference length of the papillae in the i-th sectioned image. In addition, an area of the papillae projecting onto the M-th sectioned image is measured by the analysis device 200. Next, a 3D interdigitation index is calculated by the formula:

$$I = \frac{A 1}{A 2}$$

using the analysis device 200, wherein A2 is the area of the papillae projecting onto the M-th sectioned image.

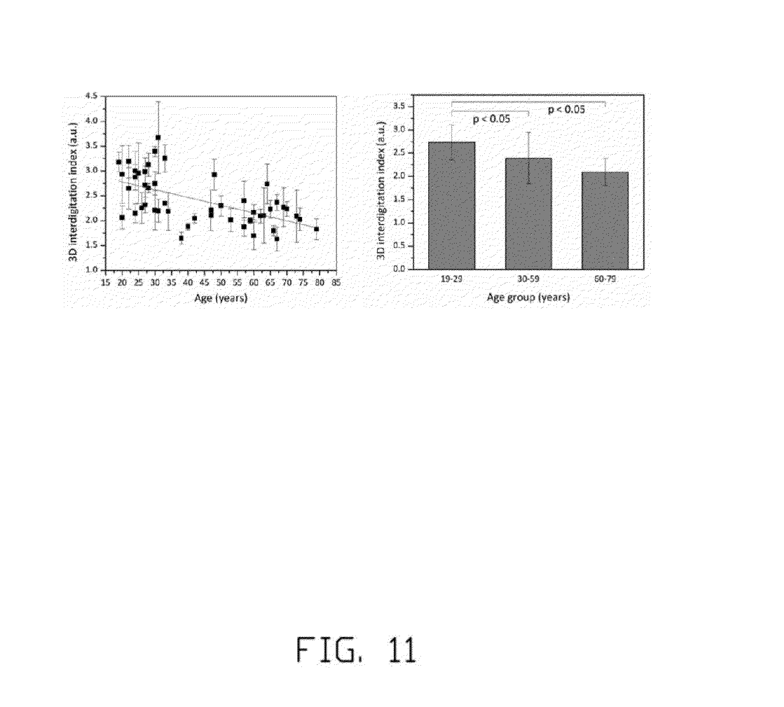

Referring to FIG. 11, statistical results show that the 3D interdigitation index decreases with aging.

4. Cellular and Nucleus Areas of Stratum Basale

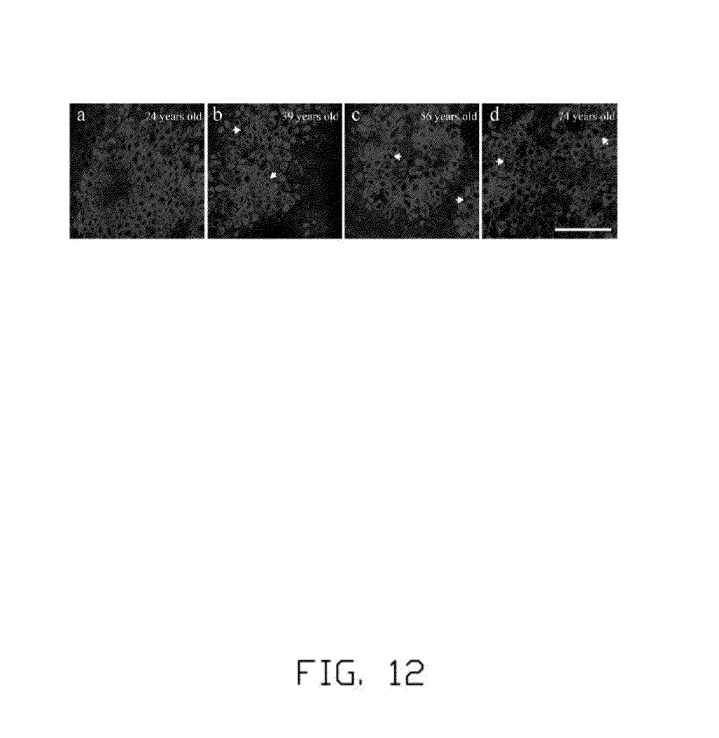

Referring to FIG. 12, for each group of sectioned images of the THG 14t or combination of the SHG 14s and the THG 14t, cells and nuclei of the stratum basale are recognized and cellular and nucleus areas of the stratum basale are measured by the analysis device 200.

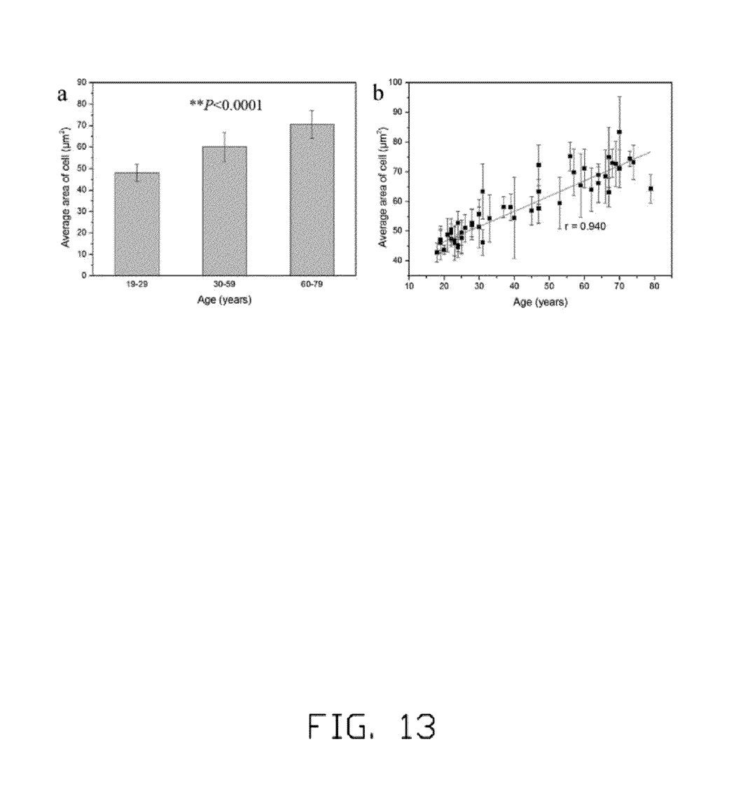

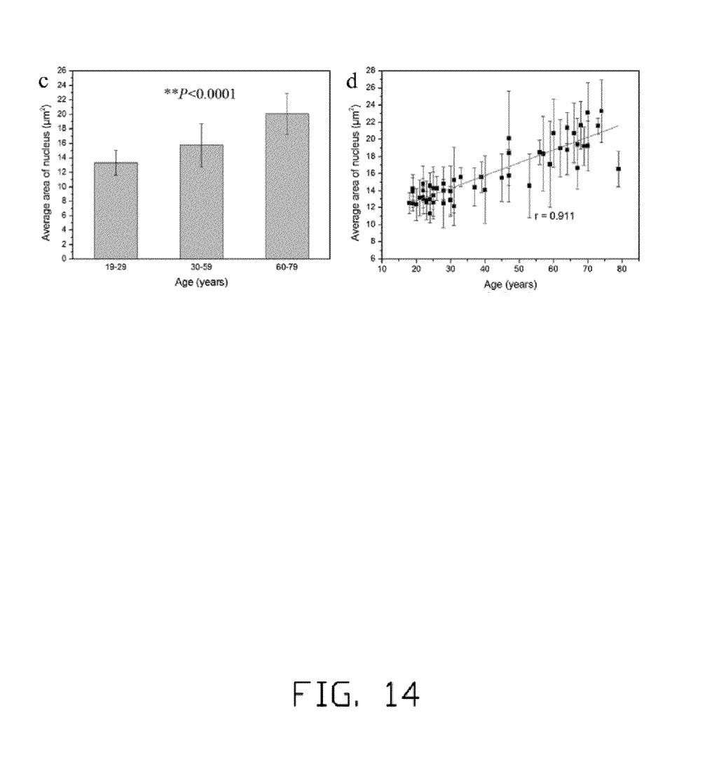

Referring to FIGS. 13-14, statistical results show that both the average cellular and nucleus areas of the skin increase with aging.

Various components of the image processing device 190 and the analysis device 200 can be integrated into a single control unit, or alternatively, can be implemented in software algorithm or hardware, such as, for example, a field programmable gate array (FPGA) or an application specific integrated circuit (ASIC). In addition, image processing and analysis method steps may be performed by the same control unit or a processor executing instructions organized into a program module or a custom designed state machine. As such, it is intended that the processes described herein be broadly interpreted as being equivalently performed by software, hardware, or a combination thereof. As previously discussed, software modules can be written, via a variety of software languages, including C, C++, Java, Visual Basic, and many others. These software modules may include data and instructions which can also be stored on one or more machine-readable storage media, such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; and optical media such as compact discs (CDs) or digital video discs (DVDs). Instructions of the software routines or modules may also be loaded or transported into the wireless cards or any computing devices on the wireless network in one of many different ways. For example, code segments including instructions stored on floppy discs, CD or DVD media, a hard disk, or transported through a network interface card, modem, or other interface device may be loaded into the system and executed as corresponding software routines or modules. In the loading or transport process, data signals that are embodied as carrier waves (transmitted over telephone lines, network lines, wireless links, cables, and the like) may communicate the code segments, including instructions, to the network node or element. Such carrier waves may be in the form of electrical, optical, acoustical, electromagnetic, or other types of signals.

In operation, the system 10 is first provided. Then the population study can be performed to establish the database. Finally, the sample 20 can be observed to obtain the corresponding group of section images. Then the analyses can be performed on the sectioned images of the sample 20 to obtain the morphological features of the sample 20 to assist to estimate a chronological age of the person.

While there have been illustrated and described what are considered to be example embodiments of the present disclosure, it will be understood by those skilled in the art and as technology develops that various changes and modifications, may be made, and equivalents may be substituted for elements thereof without departing from the true scope of the present disclosure. Many modifications, permutations, additions and sub-combinations may be made to adapt the teachings of the present disclosure to a particular situation without departing from the scope thereof. Accordingly, it is intended, therefore, that the present disclosure not be limited to the various example embodiments disclosed, but that the present disclosure includes all embodiments falling within the scope of the appended claims.

What is claimed is:

1. A system for human age estimation based upon in vivo skin imaging, the system comprising:
a harmonic generation microscopy (HGM) device configured for observing human skin and forming sectioned images of the human skin of second harmonic generation (SHG), third harmonic generation (THG), or combination of the SHG and the THG of an excitation light scanning the human skin; and
an analysis device in communication with the HGM device and configured for processing the sectioned images to reveal relationships between the morphological features of the human skin and human chronological ages.

2. The system of claim 1, wherein the relationships are stored as a database, and the analysis device is configured for processing a group of sectioned images of an in vivo sample of a person to obtain morphological features of the sample and thus, cooperating with the database, to assist chronological age estimation of the person.

3. The system of claim 1, wherein the HGM device comprises:
a laser device configured for emitting a laser beam of a predetermined wavelength, pulse width, repetition rate, and output power;
a scanning device and an objective lens device positioned along a path of the laser beam and configured for directing the laser beam as a scanning beam which functions as the excitation light and is focused into the human skin by the objective lens to form a scanning point, the scanning device and the objective lens device being configured for cooperatively moving the scanning point to perform a three-dimensional scanning operation on the human skin in an XYZ coordinate system of which a Z-direction is substantially parallel with a depth direction of the human skin, an observation beam containing SHG and THG of the excitation light being induced and reflected off;
a beam splitting device positioned in a path of the observation beam and configured for separating and directing the SHG and the THG to transmit separately;
a filtering device configured for filtering noise out of the observation beam, the SHG, and the THG;
a detecting device positioned in paths of the SHG and the THG and configured for detecting and converting the filtered SHG and the THG of each scanning point into signals; and
an image processing device in communication with the scanning device, the objective lens device, and the detecting device, the image processing device being configured for synchronizing the detecting device with the scanning device and the objective lens device, reading the signals corresponding to the SHG and THG of each scanning point, and thus plotting the sectioned images of the SHG, the THG, or combination of the SHG and the THG.

4. The system of claim 3, wherein the predetermined wavelength falls into a range from about 1200 nanometers (nm) to 1330 nm.

5. The system of claim 3, wherein the predetermined pulse width falls into a range from about 15 femtoseconds (fs) to about 500 fs.

6. The system of claim 3, wherein the predetermined repletion rate falls into a range from about 10 million hertz (MHz) to about 150 MHz.

7. The system of claim 3, wherein the predetermined output power falls into a range from about 10 milliwatts (mW) to about 1500 mW.

8. The system of claim 3, wherein the laser device comprises:
a gain medium configured for amplifying light of the predetermined wavelength;
a pump source configured for supplying energy to the gain medium in a form of electrical current or pumping light having a wavelength different from the predetermined wavelength;
an optical cavity configured for bouncing the amplified light back and forth, passing through the gain medium, such that the amplified light is amplified each time passing the gain medium and comes out as the laser beam; and
a laser controller in communication with the pump source, the gain medium, and the optical cavity and configured for controlling various parameters of the laser beam.

9. The system of claim 8, wherein the pump source is selected from the group consisting of a semiconductor laser, an ytterbium fiber laser with a wavelength at about 1060 nm, a neodymium doped yttrium aluminum garnet laser, a titanium doped sapphire laser with a wavelength at about 800 nm, and a large-mode-area ytterbium doped photonic crystal fiber (PCF) laser with a wavelength of about 1040 nm, a repetition rate of about 50 MHz, and a pulse width of about 100 fs.

10. The system of claim 8, wherein the gain medium is chromium doped forsterite.

11. The system of claim 8, wherein the optical cavity is selected from the group consisting of an optical parametric oscillator (OPO) and a PCF oscillator.

12. The system of claim 3, wherein the laser device is selected from the group consisting of:
a femtosecond chromium doped forsterite laser with the predetermined wavelength of about 1230 nm, the predetermined pulse width of about 100 fs, the predetermined repetition rate of about 110 MHz, and the predetermined output power of about 500 mW pumped by the about 1060 nm ytterbium fiber laser;
a femtosecond chromium doped forsterite laser with the predetermined wavelength of about 1260 nm, the predetermined pulse width of about 51 fs, the predetermined repetition rate of about 95 MHz, and the predetermined output power of about 800 mW pumped by the about 1060 nm ytterbium fiber laser;
a femtosecond OPO laser with the predetermined wavelength of about 1250 nm, the predetermined pulse width of 200 fs, the predetermined repetition rate of about 80 MHz, and the predetermined output power of about 580 mW, pumped by the about 800 nm titanium doped sapphire laser; and
a femtosecond laser with the predetermined wavelength of about 1260 nm, the predetermined repetition rate of about 50 MHz, the predetermined pulse width of about 100 fs, and the predetermined output power of 320 mW based on a 1040 nm, 50 MHz, 100 fs amplified output of the large-mode-area ytterbium-doped PCF oscillator-amplifier laser.

13. The system of claim 3, wherein the scanning device comprises:
a pair of mirrors positioned along the path of the laser beam;
a scanning motor coupled to the mirrors; and
a scanning controller in communication with the scanning motor and configured for controlling the scanning motors to realize fast and precise positioning of the mirrors to perform an XY-plane scanning within one of the stacks of the human skin to form a corresponding sectioned image, with various controlled parameters.

14. The system of claim 13, wherein one of the mirrors is configured for moving the scanning point along an X-direction of the XY-plane, and the other is configured for moving the scanning point along a Y-direction of the XY-plane.

15. The system of claim 13, wherein the scanning motor is based on moving magnet technology.

16. The system of claim 13, wherein the various controlled parameters comprises a scanning range of the XY-plane, step sizes in the X-direction and the Y-direction, and a scanning speed.

17. The system of claim 3, wherein the scanning device has a schema selected from the group consisting of a galvo pair scanning mirrors schema and a galvo-resonance scanning mirrors schema.

18. The system of claim 3, wherein the objective lens device comprises:
a tube lens and a objective lens positioned along a path of a rotating beam from the scanning beam, the tube lens being configured for diverging a rotating beam into a light cone, the objective lens being configured for focusing the light cone into the human skin;
an objective motor coupled to the objective lens; and
an objective controller in communication with the objective motor and configured for controlling the objective motor to move the objective lens along a direction that is substantially parallel with the Z-direction, thus moving the scanning point along the Z-direction to form the sectioned images of the different stacks of the human skin, with various controlled parameters.

19. The system of claim 18, wherein the objective lens is an infrared water immersion objective lens.

20. The system of claim 18, wherein the various controlled parameters comprises a step size in the Z-direction and a scanning depth, the step size in the Z-direction is about 5 um, and the scanning depth is about 300 um.

21. The system of claim 3, wherein the beam splitting device comprises:
a first dichromatic beam splitter (DBS) positioned in the path of the objective lens device for passing the laser beam but deflecting the observation beam; and
a second DBS positioned in the path of the observation beam reflected off from the first DBS and configured for passing the SHG directly but deflecting the THG.

22. The system of claim 21, wherein the first DBS works at about 850 nm and the second DBS works at about 490 nm and both are about 45-degree tilted in relative with a transmitting direction of the observation beam.

23. The system of claim 21, wherein the filtering device comprises:
a first filter positioned between the first DBS and second DBS and configured for filtering the noise out of the observation beam;
a second filter positioned in the path of the SHG between the second DBS and the detecting device and configured for filtering the noise out of the SHG; and
a third filter is positioned in the path of the THG between the second DBS and the detecting device and configured for filtering the noise out of the THG.

24. The system of claim 23, wherein the first filter is a color filter that works at wavelengths from about 400 nm to about 700 nm, the second filter and the third filter are both band-pass filters that respectively work at wavelengths from about 600 nm to about 650 nm, and wavelength from about 400 nm to about 430 nm.

25. The system of claim 3, wherein the detecting device comprises:
a first detector positioned in the path of the SHG from the filtering device and configured for detecting and converting the SHG of each scanning point into corresponding signals; and
a second detector positioned in the path of the THG from the filtering device and configured for detecting converting the THG of each scanning point into corresponding signals.

26. The system of claim 25, wherein the first detector and the second detector are photomultiplier tubes.

27. The system of claim 3, wherein the signals are electric signals selected from the group consisting of electric charges, currents, and voltages of values that are proportional to intensities of the corresponding SHG and the corresponding THG.

28. The system of claim 3, wherein the HGM device comprises a beam shaping device positioned in a path of the laser beam and configured for shaping the laser beam into a shaped beam having a shape, size, and collimation degree complying with requirements of the scanning.

29. The system of claim 3, wherein the HGM device comprises a holding device positioned adjacent to the object lens device and configured for holding and stabilizing the human skin.

30. The system of claim 29, wherein the holding device comprises a substantially disk-like main body, and a vacuum source, the main body defines a substantially circular observation window through a substantially central portion thereof, the main body also defines a plurality of vents surrounding the window, the scanning beam projects from the window, the vents are communicated with the vacuum source such that the human skin is vacuum-held by the vacuum source through the vents and is exposed to the scanning beam via the window after being put on the main body.

31. The system of claim 1, wherein the system is configured for performing a population study on a plurality of subjects of different ages, each subject is observed by the HGM device to obtain a group of sectioned images, the analysis device is configured for recognizing each papilla in each group of sectioned images by processing the sectioned images, in this order from a superficial layer to a deep layer of human skin, to find the n-th sectioned image in which the papilla is first present and the m-th sectioned image in which the papilla is first vanished, and the analysis device is also configured for calculating a height of the papilla by the formula: H=^{z}*|(m−n)|, wherein H is the papilla height and z is the step size of the scanning in the Z-direction.

32. The system of claim 1, wherein the system is configured for performing a population study on a plurality of subjects of different ages, each subject is observed by the HGM device to obtain a group of sectioned images, the analysis device is configured for recognizing each papilla in each group of sectioned images by processing the sectioned images, in this order from a superficial layer to a deep layer of human skin, to find the n-th sectioned image in which the papilla is first present and the m-th sectioned image in which the papilla is first vanished, and the analysis device is also configured for calculating a volume of the papilla by the formula:
$$V = {\sum }_{i = m}^{i = n} z * {s}_{i} ,$$
wherein V is the volume and sᵢ is the area of the papilla in the i-th sectioned image, and n≦i≦m.

33. The system of claim 1, wherein the system is configured for performing a population study on a plurality of subjects of different ages, each subject is observed by the HGM device to obtain a group of sectioned images, the analysis device is configured for recognizing each papilla in each group of sectioned images by processing the sectioned images, in this order from a superficial layer to a deep layer of human skin, to find the M-th sectioned image in which all the papillae are first vanished, and the analysis device is also configured for calculating a thickness of the epidermis including the papillae by the formula: T=M*z, wherein T is the thickness of the epidermis including the papillae and M is a positive integer.

34. The system of claim 1, wherein the system is configured for performing a population study on a plurality of subjects of different ages, each subject is observed by the HGM device to obtain a group of sectioned images, the analysis device is configured for recognizing each papilla in each group of sectioned images by processing the sectioned images, in this order from a superficial layer to a deep layer of human skin, to find the N-th sectioned image in which any papilla is first present and the M-th sectioned image in which all the papillae are first vanished, the analysis device is configured for calculating an interface area by the formula:
$$A 1 = {\sum }_{i = M}^{i = N} z * {L}_{i} ,$$
wherein Lᵢ is a circumference length of the papillae in the i-th sectioned image, the analysis device is configured for measuring an area of the papillae projecting onto the M-th sectioned image, and the analysis device is also configured for calculating a 3D interdigitation index by the formula:
$$I = \frac{A 1}{A 2} ,$$
wherein A2 is the area of the papillae projecting onto the M-th sectioned image.

35. The system of claim 1, wherein the system is configured for performing a population study on a plurality of subjects of different ages, each subject is observed by the HGM device to obtain a group of sectioned images, the analysis device is configured for recognizing cells and nuclei of the stratum basale, and the analysis device is configured for measuring cellular and nucleus areas of the stratum basale.

36. A method for human chronological age estimation based on in vivo skin imaging, the method comprising:
providing a system comprising a HGM device and an analysis device in communication with the HGM device;
observing human skin of a study population comprising a plurality of subjects to obtain a plurality groups of sectioned images using the HGM device, each group of sectioned images being taken from the human skin of a subject and corresponding to SHG, THG, and combination of the SHG and THG of an excitation light scanning the human skin; and
processing each group of sectioned images to reveal relationships between the morphological features of the human skin and human chronological ages.

37. The method of claim 36, further comprising:
storing the relationships as a database;
observing a sample of the skin of a person to obtain a group of sectioned images;
processing the section images of the samples to obtain morphological features of the skin of the person; and
estimating a chronological age of the person with assistance of the morphological features of the skin of the person and the database.

38. The method of claim 36, wherein the sectioned images are taken from the human skin whose aging is mainly caused by intrinsic factor.

39. The method of claim 38, wherein the human skin is a skin of forearm, leg or abdomen.

40. A method for examining a human skin in vivo for determining a chronological aging degree thereof to estate an age of a human being having the human skin, comprising:
using a HGM device microscopy to check the human skin by scanning a laser beam to the human skin, wherein the laser beam is excited by the human skin to generate an SHG and a THG;
using photomultiplier tubes to convert the SHG and THG into respective electrical signals;
using an analysis device to analyze the electrical signals to obtain a morphological feature of the human skin and to decide the aging degree of the human skin in accordance with the morphological feature of the human skin.

41. The method of claim 40, wherein the morphological feature is related to one of following features: an average of isolated dermal papilla volume, an average of isolated dermal papilla height, an average area of cell, an average area of nucleus and an average thickness of viable epidermis.

42. The method of claim 41, wherein the laser beam is generated by a 1230 nm femtosecond Cr—F laser.

43. The method of claim 41, wherein the skin in vivo is held in a stable condition by a holding device which uses a vacuum to suck the skin in vivo in position.

44. The method of claim 41, wherein the laser beam is generated by a 1230 nm femtosecond Cr—F laser having an output power of 500 mW and the scanning of the laser to the skin in vivo is lasted for at most 30 minutes.
